(19) United States
(12) Patent Application Publication
(10) Pub. No.: US 2003/0045835 A1
(43) Pub. Date: Mar. 6, 2003

(54) Method and apparatus for coagulation and closure of pseudoaneurysms

(75) Inventors: Edward J. Anderson, Hopkins, MN (US); James V. Kauphusman, Champlin, MN (US); Mark B. Saker, Chicago, IL (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Vascular Solutions, Inc.

(21) Appl. No.: 09/943,584

(22) Filed: Aug. 30, 2001

(51) Int. Cl. A61M 31/00

(52) U.S. Cl. 604/164.01

(57) ABSTRACT

Safe and convenient methods and devices for inexpensively and rapidly treating pseudoaneurysms and other subcutaneous pools of blood utilize an apparatus with an applicator that can be grasped by a user at its proximal end and which has a distal portion that can be percutaneously inserted into a pseudoaneurysm. The distal portion has at least one retaining structure on at least one surface; the retaining structure(s) serve to collectively retain a clotting agent as the distal portion is inserted into the pseudoaneurysm. The clotting agent in the retaining structure(s) is exposed to blood in the pseudoaneurysm and initiates a clotting cascade. Since the clotting agent is in the retaining structure(s) and is not forcibly injected into the blood, the clotting cascade causes blood to clot at or near the surface of the applicator. The clot is associated with the applicator until the distal portion of the applicator is removed from the pseudoaneurysm. Removing the applicator causes the patient's tissue to wipe the clot off of the applicator so that the clot remains in the pseudoaneurysm.

### FIELD OF THE INVENTION

The invention relates to the repair and closure of wounds and abscesses, especially pseudoaneurysms. More particularly, the invention relates to the percutaneous insertion of a clotting agent for the coagulation and closure of pseudoaneurysms caused by arterial entry as part of endoscopic treatment.

### BACKGROUND OF THE INVENTION

Numerous interventional medical procedures are performed by inserting medical instruments into patients via punctures in an artery. As a part of these procedures, patients are typically treated with anticoagulant agents that slow the clotting of their blood. When the instruments are removed, it is usual to apply pressure to the punctured artery to prevent bleeding. The presence of the anticoagulating agent increases the time that is required for the puncture to be sealed.

One complication of these procedures is the formation of a pseudoaneurysm. A pseudoaneurysm is formed when blood seeps from the patient's blood vessel through the puncture site and forms a turbulent pool of unclotted blood underneath the skin. Due to the turbulence, the blood in the pseudoaneurysm fails to clot. The frequency of pseudoaneurysms is a problem that has increased in recent years because of the increased popularity of such procedures, the use of larger instruments, and the increased use of blood thinning drugs. The conventional form of treatment for pseudoaneurysms is to apply continuous pressure over the site of the injury to stop the turbulence and allow the blood to clot. Patients' compression times vary from minutes to hours resulting in discomfort to the patient and, in the case of manual compression, to the caregiver. Following compression, the caregiver must typically verify the procedure's success by ultrasound. Oftentimes, compression is unsuccessful and surgical repair is required.

One treatment for aneurysms and pseudoaneurysms is to deliver an expanding occlusive device that anchors inside the aneurysm. The occlusive device serves as a foreign body that initiates blood clotting around the foreign body. Examples of these types of occlusive devices and anchors are shown in U.S. Pat. Nos. 5,649,959, 6,063,070, 6,063,104, 6,139,564, 6,187,024 and 6,193,708. While effective, the disadvantage of using an occlusive device is that the occlusive device usually remains in the body. In the context of aneurysms deep in the body this does not present a serious problem, but in the case of pseudoaneurysms that are just under the skin and typically on the inside of the leg, the remnants of a foreign body under the skin may not be acceptable. The foreign body also carries a risk of possible infection.

An alternative pseudoaneurysm treatment is to use a syringe or needle to inject a blood clotting agent directly into the pseudoaneurysm. Examples of this technique are described in Cope et al., Coagulation of Aneurysms by Direct Percutaneous Thrombin Injection, 147 American Journal of Roentgenology, 383-87 (1986), and U.S. Pat. No. 5,403,278. Once injected, the free-floating blood clotting agent reacts with the blood to form a clot that ultimately coagulates the pooled blood and seals the puncture site. Unfortunately, one of the inherent risks associated with direct injection of a clotting agent is that some of the clotting agent may be injected into the artery and cause small blood clots to form in the artery. These small clots can be swept away by the blood flowing in the artery and may subsequently become stuck in smaller blood vessels and block them. Clots that are stuck in the brain and block blood flow can cause strokes, while clots in the coronary arteries can result in myocardial infarction.

Various attempts have been made to improve the conventional needle or syringe for injecting a medicament into a treatment site. U.S. Pat. Nos. 4,402,308, 4,578,061, 4,700,692, 5,129,882, 5,358,474, 5,562,613, and 5,906,599 describe needles or cannulas that have a mechanism for laterally ejecting a pellet or medicament once the device is inserted. U.S. Pat. Nos. 5,310,407, 5,443,481, 5,503,623, 5,766,157, 5,814,066, and 6,010,495 describe catheter-based devices for delivering medicaments or sealants through a catheter introduced into the vascular system of the patient. U.S. Pat. Nos. 4,411,657, 4,710,180, 4,790,830, 5,290,267, 5,628,734, 5,848,996, and 6,200,302 describe needles that are curved or that have a lateral-facing port for ejecting medicaments. U.S. Pat. Nos. 4,950,234, 6,027,471, and 6,059,749 describe different syringe arrangements for injecting sealants. In each of these devices, however, the pellet or medicament is still injected or ejected such that the possibility of having a clotting agent migrate into an artery remains. It would be desirable to provide a treatment that causes the blood in pseudoaneurysms to clot without creating a risk that clotting agents will be introduced into the blood stream. An ideal treatment would be safe, inexpensive, easily administered, and fast-acting.

### SUMMARY OF THE INVENTION

The current invention provides safe and convenient methods and devices for inexpensively and rapidly treating pseudoaneurysms and other pools of blood within the body. The invention includes an apparatus with an applicator that can be grasped by a user at its proximal end and which has a distal portion that can be percutaneously inserted into a pseudoaneurysm. The distal portion has at least one retaining structure on at least one surface; the retaining structure(s) serve to collectively retain a clotting agent as the distal portion is inserted into the pseudoaneurysm. The clotting agent in the retaining structure(s) is exposed to blood in the pseudoaneurysm and initiates a clotting cascade. Since the clotting agent is in the retaining structure(s) and is not forcibly injected into the blood, the clotting cascade causes blood to clot at or near the surface of the applicator. The clot is associated with the applicator until the distal portion of the applicator is removed from the pseudoaneurysm. Removing the applicator causes the patient's tissue to wipe the clot off of the applicator so that the clot remains in the pseudoaneurysm.

In use, the retaining structure(s) of the applicator are associated with the clotting agent, for example, by dipping the retaining structure(s) into a solution of thrombin. The user creates an entry for introducing at least a portion of the distal portion of the applicator into the pseudoaneurysm and introduces at least a portion of the distal portion of the applicator into the pseudoaneurysm. The user thereby exposes at least a portion of the clotting agent to blood in the pseudoaneurysm. The clotting agent initiates a clotting cascade such that at least one clot is operably attached to the applicator. The user then waits until the clotting cascade has progressed sufficiently to make the clot larger than the entry and withdraws the applicator from the pseudoaneurysm so as to leave the clot within the pseudoaneurysm.

One embodiment of the invention includes an apparatus with at least one spring member associated with the distal portion of the applicator. The spring member has coils that have spaces between them. These spaces may be used as a retaining structure for the clotting agent. The spring is introduced into the pseudoaneurysm and the clotting agent initiates a clotting cascade. Another embodiment includes an outer sleeve that is coaxial with at least one of the spring members and a plunger member that is operably engaged with the spring member(s). The plunger member is used to selectively extend the at least one spring member beyond the distal end of the outer sleeve so that the clotting agent is exposed to the blood. Alternatively, the plunger member is used to retract the outer sleeve to expose the spring member and the clotting agent to the blood. The clotting agent remains on or near the spring member as it is exposed to the blood.

The clotting agent need only be applied or deposited onto the applicator of the invention. When the clotting agent is exposed to the blood, it remains at or near the applicator as it reacts with the blood. The clotting agent thus maintains a safe distance from the patient's blood vessels. Even in a turbulent blood flow, it has been observed that the clot sticks to and remains otherwise associated with the preferred embodiment of the applicator. In contrast, a prior art method of injecting clotting agents into the blood separates the clotting agent from the injection apparatus with the result that the clotting agent does not remain near the injection apparatus. Moreover, the injection process uses a highly concentrated stream of clotting agent that can exit a pseudoaneurysm without being reacted and enter the patient's blood stream to cause an unwanted clot.

An embodiment of the invention includes a retaining structure with a roughened, irregular surface that facilitates retention of the wettable clotting agent. Alternatively, the retaining structure is a roughened, irregular surface that holds wet or dry clotting agents. Embodiments of the retaining structure also include an applicator with a notch or an indentation, or a plurality of notches or indentations. The notches or indentations may be configured to hold large amounts of material, for example several milliliters of fluid or several tenths of a gram of a solid, or to accept only very small amounts of material, for example, only a fraction of a microgram of solid or fractions of a microliter of liquid. Embodiments include retaining structures that have smaller retaining structures within them.

Another embodiment of the invention includes an apparatus that has an applicator and a stylet. The stylet is adapted to create an entry in a patient's skin that is used by the applicator. The stylet may be integral with the applicator or a separate part. In one embodiment, the stylet is a sharp tip on the applicator. In another embodiment, the stylet is a sharp edge on an introducer that is used to create an entry for the applicator. A user manipulates the stylet to create an entry in a patient's skin through which the applicator is introduced.

A clotting agent for use with the present invention may be a liquid or powder. Many materials are known to initiate clotting and these materials may be used as a substitute for the clotting agent or in combination with the clotting agent. Such clot initiating materials include enzymes or polymers that occur in nature and synthetic materials that have been observed to initiate blood clotting.

### BRIEF DESCRIPTION OF THE DRAWINGS

FIG. 1 is a perspective view of an embodiment of the invention.

FIG. 2 is a perspective view of a preferred embodiment of the invention.

FIG. 3 is an enlarged, fragmentary, plan view of the distal end of FIG. 2.

FIG. 4A is a perspective view depicting a step of loading the applicator of FIG. 2.

FIG. 4B is a perspective view depicting a step of loading the applicator of FIG. 2.

FIG. 5 is a fragmentary, plan view of the distal end of an embodiment of the invention.

FIG. 6 is a fragmentary, plan view of the distal end of another embodiment of the invention.

FIG. 7 is a plan view of another embodiment of the invention.

FIG. 8 is a fragmentary, plan view of the distal end of another embodiment of the invention.

FIG. 9 is a fragmentary, plan view of the distal end of another embodiment of the invention.

FIG. 10 is a fragmentary, plan view of the distal end of another embodiment of the invention.

FIG. 11 is a fragmentary, plan view of the distal end of another embodiment of the invention.

FIG. 12 is a fragmentary, plan view of the distal end of another embodiment of the invention.

FIG. 13 is a fragmentary, plan view of the distal end of another embodiment of the invention.

FIG. 14 depicts a cross-sectional view of a pseudoaneurysm within a patient's body.

FIG. 15 is a perspective view of a kit in accordance with a preferred embodiment of the invention.

### DETAILED DESCRIPTION OF PREFERRED EMBODIMENTS

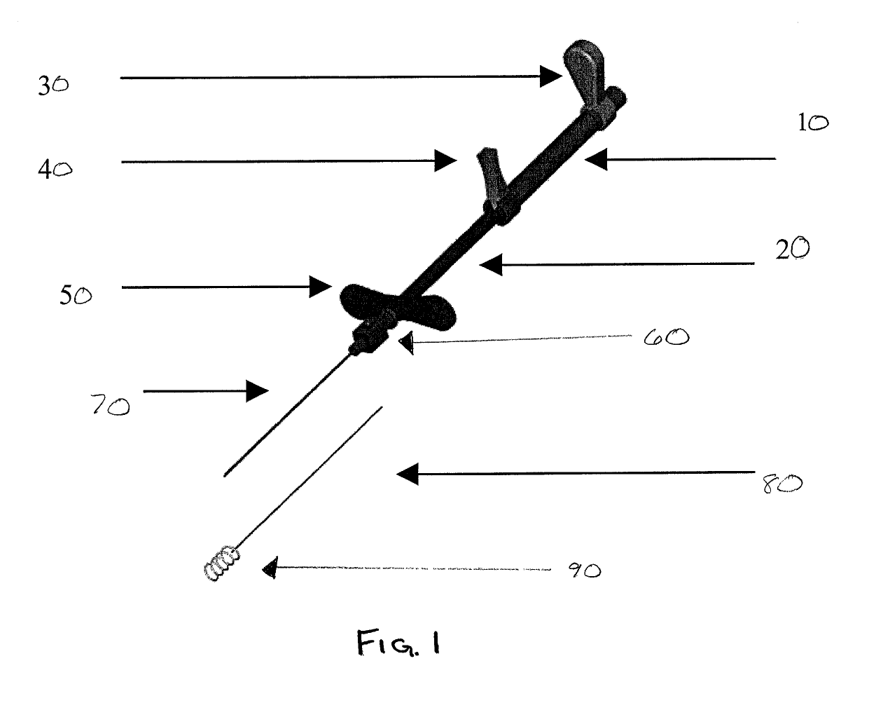

A perspective view of a preferred embodiment of the invention is shown in FIG. 1. The invention includes proximal shaft 10. Attached to proximal shaft 10 is mid shaft 20. Proximal slide 30 is located on the proximal end of the proximal shaft 10. Distal slide 40 is located on the distal end of the proximal shaft 10. Attached to the distal end of mid shaft 20 is finger wing 50. Luer tip 60 is attached to the distal end of the mid shaft 20. Attached to the distal side of luer tip 60 is hollow needle 70. Piston 80 resides within hollow needle 70. Distal projection 90 is depicted as projecting from the distal end of piston 80.

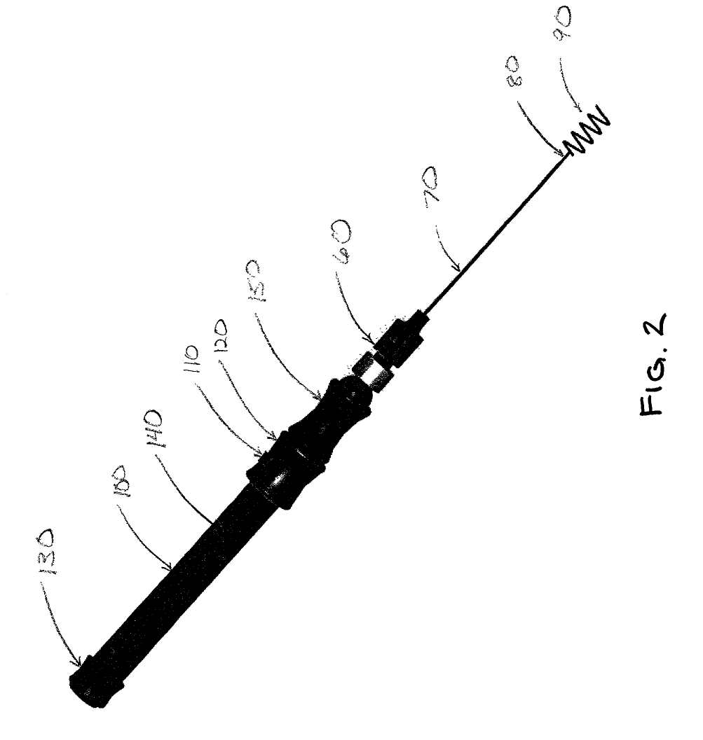

A perspective view of another embodiment of the invention is shown in FIG. 2. The invention contains piston housing 100 with slidable piston handle 110. Slidable piston handle 110 is mounted on piston handle grip 120. At the proximal end of piston housing 100 is piston body cap 130. Piston handle channel 140 runs the length of piston housing 100. At the distal end of piston housing 100 is luer tip 60. Attached to the distal side of luer tip 60 is hollow needle 70. Residing within and protruding from hollow needle 70 is piston 80. Distal projection 90 is depicted as projecting from the distal end of piston 80.

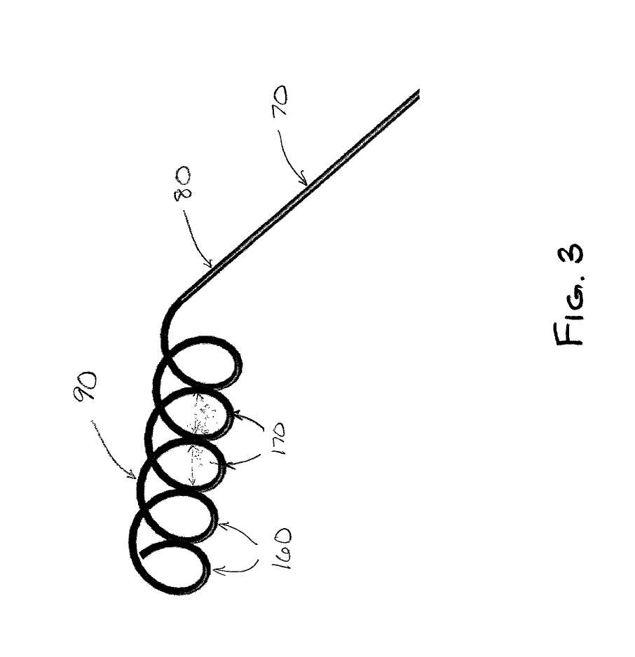

FIG. 3 depicts a fragmentary view of the distal end of the embodiments of FIGS. 1 and 2. Extending out of hollow needle 70 is piston 80. At the distal tip of piston 80 is distal projection 90. Distal projection 90 is preferably a spring member comprised of a coiled wire that preferably pigtails into loops 160 once deployed. In one embodiment, the distal projection 90 is formed of superelastic Nitinol. In alternative embodiments, the distal projection 90 is made of silica, copper, gold or other suitable thrombogenic materials. In one embodiment, the length of distal projection 90 is 4 inches long but the length can vary according to application up to 12 inches long, although longer lengths may be used for other embodiments. Preferably, the pitch of distal projection 90 is such that small interstitial spaces 170 are formed between adjacent coils of the distal projection 90. Pitches of the distal projection 90 can preferably range between 0.015 to 0.036 inches. Similarly, the preferred diameter of the distal projection 90 can range between 0.010 inches to 0.040 inches.

It will be recognized that both the interstitial spaces 170, as well as the larger structure of the loops 160 serve to define retaining structures for purposes of the present invention. In one embodiment, the pitch of distal projection 90 is sized to provide a holding capacity of 100-400 US units of thrombin per inch. In alternative embodiments, other suitable thrombogenic materials are used in place of thrombin. Distal projection 90 can be provided in either a thrombogenically pre-coated or uncoated form. As will be described, other alternative retaining structures can also be utilized to accomplish the retention function of the clotting agent in accordance with the present invention. In one embodiment, distal projection 90 may receive a surface treatment such as roughening treatment with a hydrophilic substance to facilitate capture and retention of the thrombogenic material. In addition, alternative embodiments may utilize a packing mechanism through which thrombin or appropriate thrombogenic materials can be imparted to a retaining structure.

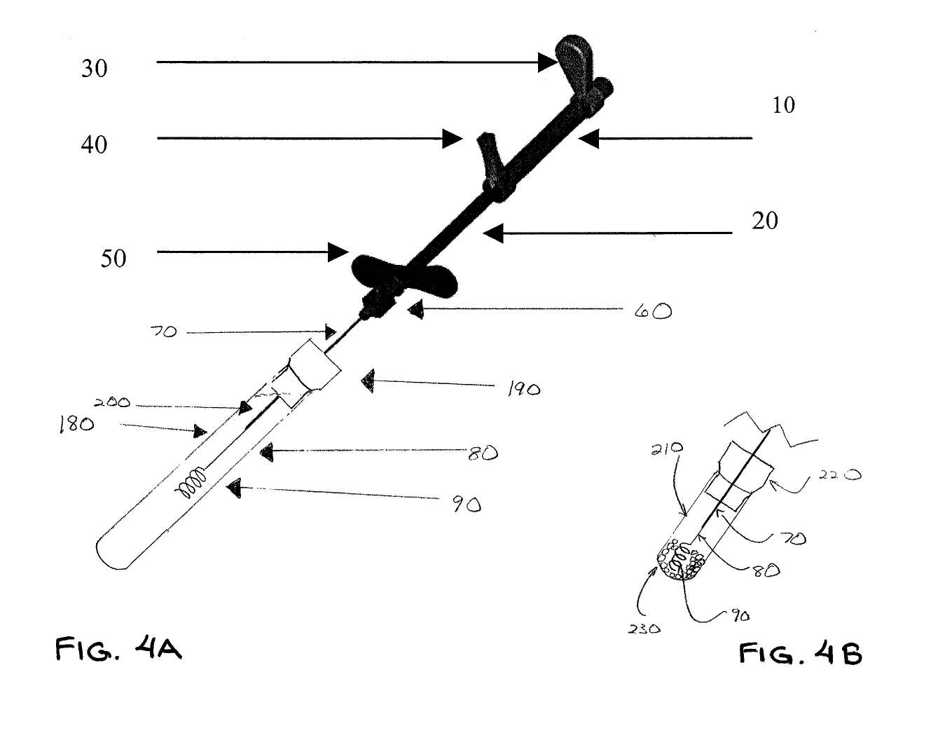

FIGS. 4A and 4B depict perspective views of the hemostatic capture apparatus of the invention as shown in FIG. 1 that is preferably used in a two step process of loading the clotting agent 230 onto the applicator as will be described. At the distal end of the invention is wetting agent vial 180. Rubber septum 190 is located in the open end of wetting agent vial 180. A liquid wetting agent 200 is located inside the wetting agent vial 180. Dry clotting agent vial 210 is also shown attached to the distal end of the invention. Rubber septum 220 is preferably located in the open end of dry clotting agent vial 210. Dry clotting agent 230 is located inside dry clotting agent vial 210.

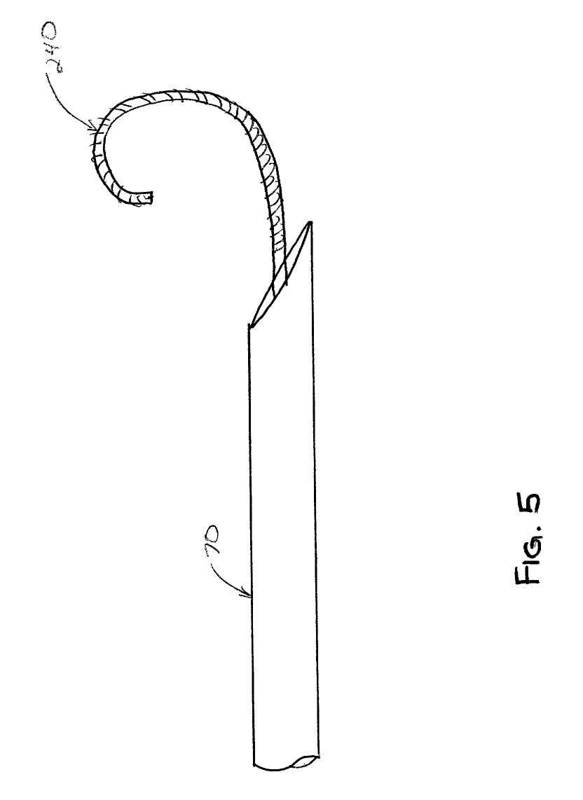

FIG. 5 contains a fragmentary, plan view of the distal end of an embodiment of the invention. In this embodiment, a wire applicator 240 extends from hollow needle 70 to form the retaining structures in accordance with the present invention. In this embodiment, the coiled wire applicator is not configured to establish a series of loops when extended. The details of the construction, arrangement, and pitch of the coiled wire are preferably similar to those described with respect to the embodiment shown in FIG. 3, although other configurations could be adopted as long as a desired quantity of clotting agent 230 was able to be retained by the applicator. For example, multiple separate coiled wire applicators 240 could be extended from hollow needle 70.

For purposes of the present invention, the retaining structure(s) are intended to define a sufficient volume of space for retaining an aliquot of clotting agent 230 associated with the applicator 240 during the procedure as will be described. The amount of clotting agent 230 necessary for a given medical procedure would depend upon the nature of the procedure and the particular clotting agent 230. For example, if the clotting agent 230 is dry powdered thrombin, and the wetting agent 200 is saline, it is preferred that between 100 and 400 units of thrombin are effectively retained per inch by the retaining structures 240. In a preferred embodiment, coiled wire applicator 240 is approximately 4 inches long such that between about 400 and 1600 units of thrombin are introduced in order to treat a typical pseudoaneurysm created as a result of a vascular access procedure and having a blood pool of at least about 2 centimeters in diameter and preferably having a volume ranging between about 4 cc and 65 cc. Existing medical literature indicates that most pseudoaneurysms can be resolved by injection of between 100 to 1500 units of thrombin.

Although the preferred embodiment of the present invention has been described with respect to a wetting process for adhering or otherwise associating the clotting agent with the applicator, it will be understood that alternative techniques may also be used. For example, the clotting agent could be provided in a gelatinous or semi-liquid or tacky form so that the clotting agent would stick to the retaining structures without the need for wetting the applicator. Alternatively, the clotting agent could be attracted to the applicator by application of electrostatic charge.

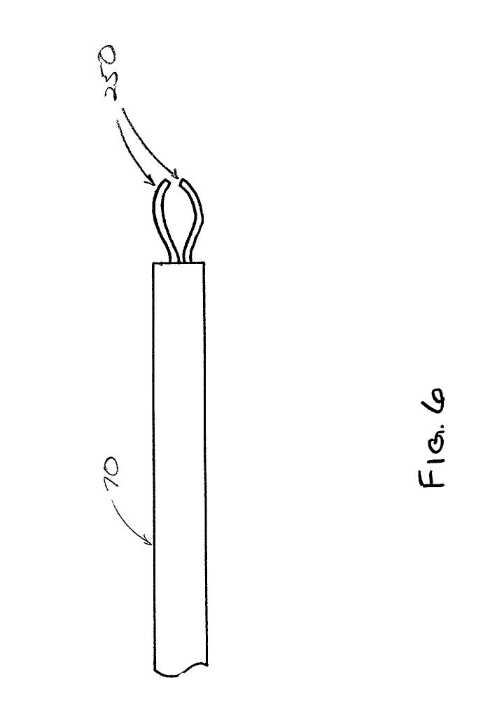

FIG. 6 contains a fragmentary, plan view of the distal end of another embodiment of the invention. In this embodiment, a set of retractable fingers 250 extend from hollow needle 70. Although only a pair of fingers 250 are shown, it will be understood that numerous arrangements of the number and configurations of such fingers 250 could be accomplished within the scope of 20 the present invention. Preferably, the retractable fingers 250 are arranged to hold larger clumps of the clotting agent 230. Alternatively, more fingers 250 could be arranged in closer parallel arrangement to one another to provide retaining structure for smaller sizes of clotting agent. In another embodiment, the retaining fingers 250 can be utilized to secure an intermediate carrier structure, such as a sponge or structure having a porous surface, that is carried by the retaining fingers 250 and holds the clotting agent 230.

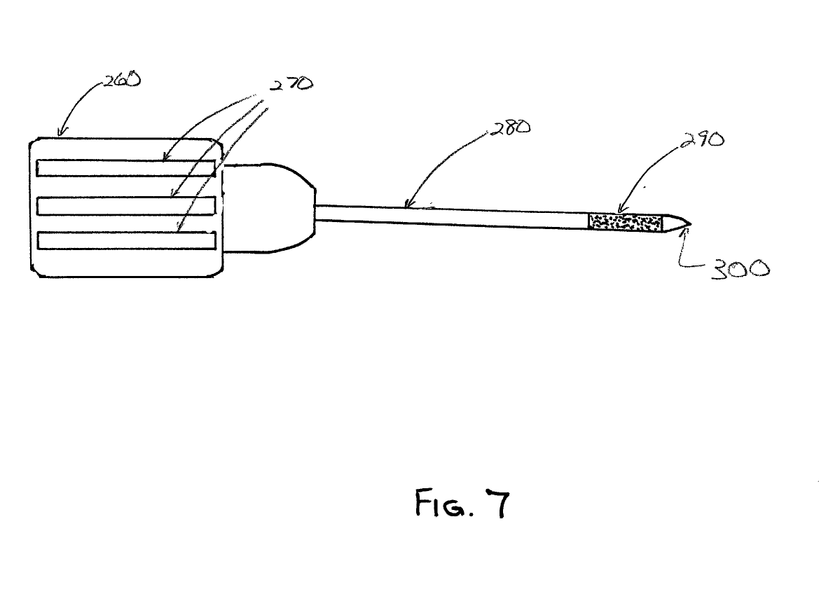

FIG. 7 is a plan view of another embodiment of the invention. On the proximal end is stylus handle 260 with multiple grooved handle grips 270. Applicator 280 is attached to the distal end of stylus handle 260. Near the distal end of applicator 280 are defined retaining structures 290. In this embodiment, the retaining structures 290 can be created by defining a roughened surface portion of the applicator 280, or more preferably, by creating a textured region having surface structure which will retain the clotting agent 230, particularly after the distal end of applicator 280 has been wetted. In this embodiment, applicator 280 is a convex point stylet tip 300 that serves as a stylet for creating a puncture to access the pseudoaneurysm.

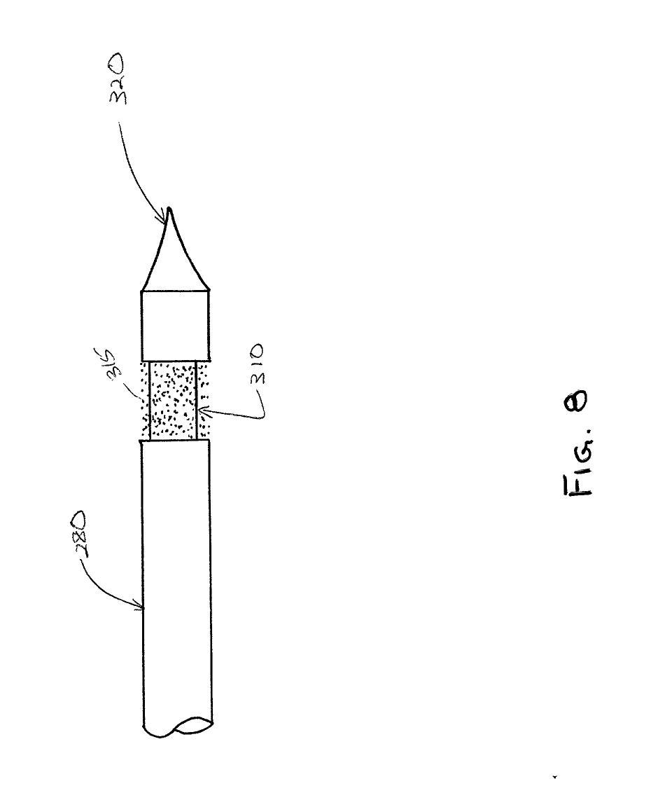

FIG. 8 shows an alternative embodiment of the invention. Near the distal end of the applicator 280 is a recessed clotting agent retaining structure 310. The recessed retaining structure 310 has a cross-sectional diameter that is smaller than the cross-sectional diameter of application 280. Preferably, micro structures 315 are provided in the recess 310 that serve to enhance the ability of the recess 310 to retain the clotting agent 230. Such micro structures 315 can include porous coatings, abrasions, indentations, scorings, filaments, or fabric structures or the like. At the distal end of the applicator 280 is a concave point stylet tip 320.

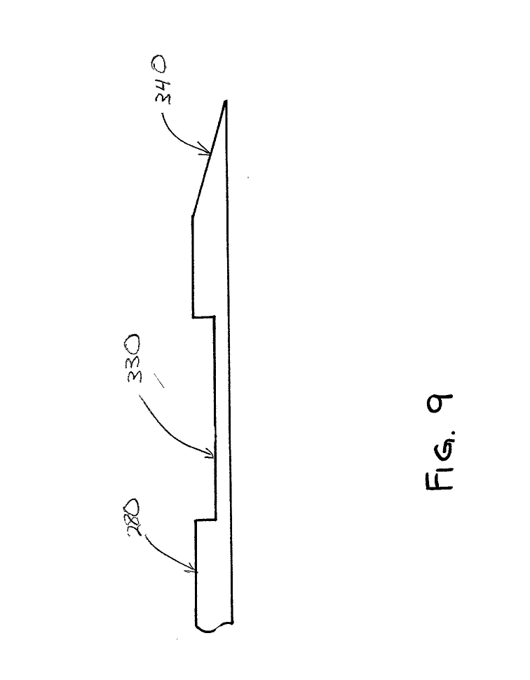

FIG. 9 shows an alternative embodiment of the applicator of the invention. Near the distal end of applicator 280 is at least one indented retaining structure 330. Indented retaining structure 330 can also be provided with micro structures to enhance the retention of clotting agent 230. At the distal end of applicator 280 is a chiseled stylet tip 340. Preferably, indented retaining structure 330 is formed of a cutaway portion that leaves sufficient structural integrity to the remainder of the applicator 280 to allow stylet tip 340 to be effectively used to create a puncture.

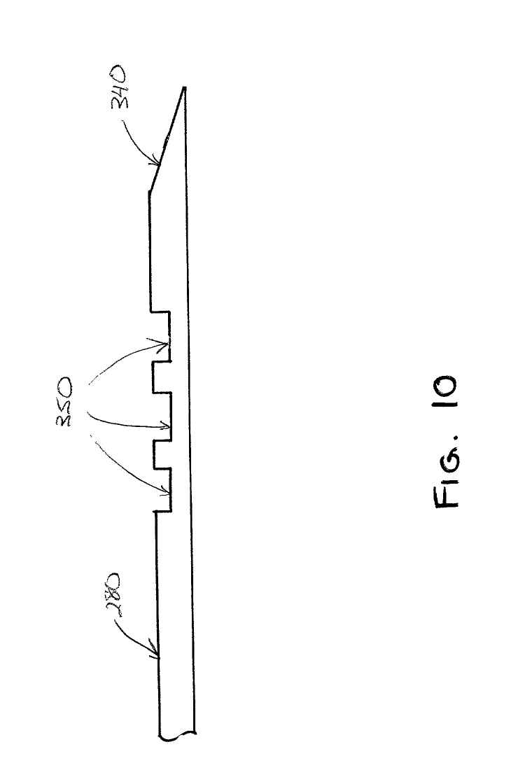

FIG. 10 shows an alternative embodiment of the invention. Near the distal end of applicator 280 are multiple indented retaining structures 350 defined along a longitudinal axis of the applicator 280. Like the indented retaining structure 330, the multiple indented retaining structures 350 can also be provided with micro structures to enhance the retention of clotting agent 230. At the distal end of applicator 280 is a chiseled stylet tip 340.

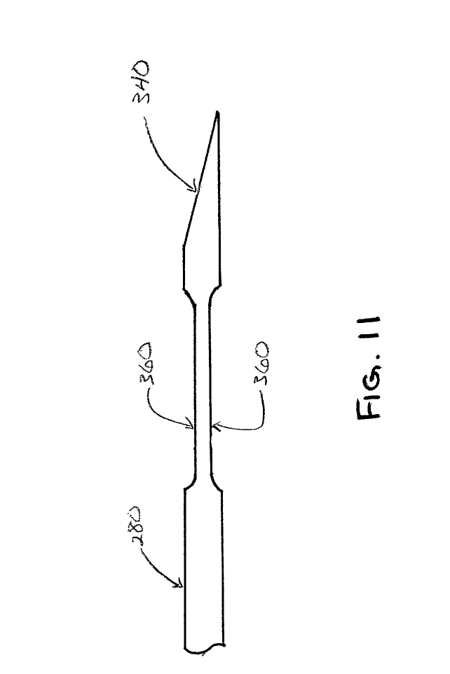

FIG. 11 shows an alternative embodiment of the invention. Near the distal end of applicator 280 are at least two recessed clotting agent retaining structures 360 defined along a common longitudinal portion of applicator 280. Like the indented retaining structure 330, the parallel recessed retaining structures 360 can also be provided with micro structures to enhance the retention of clotting agent 230. At the distal end of the applicator 280 is a chiseled stylet 340.

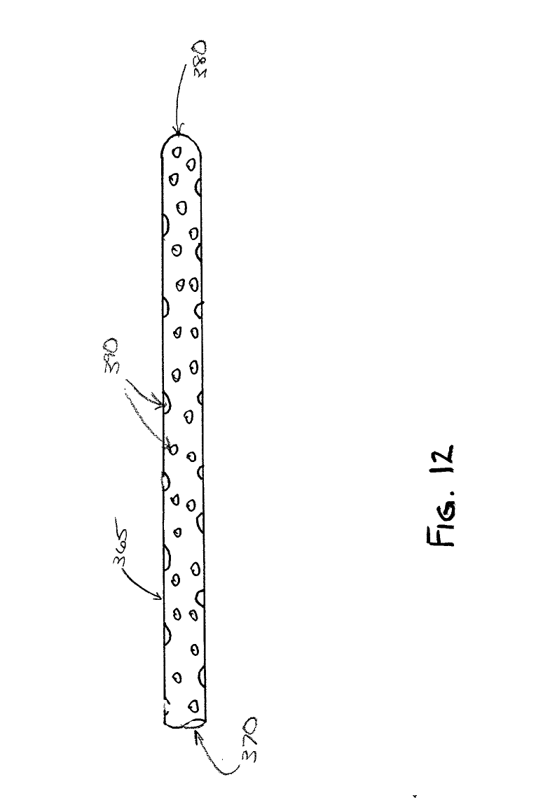

FIG. 12 shows an alternative embodiment of the invention. Applicator 282 has a plurality of delivery perforations 390 defined around an exterior of the distal tip. The delivery perforations or indentions 390 can be defined in any number of sizes and shapes. In one embodiment, the applicator 282 is a hollow tube and the perforations can extend from the surface of the applicator 282 through to a central lumen 270. In this embodiment, the distal tip is a spherical rounded end 282 that is not equipped to be utilized as a stylet.

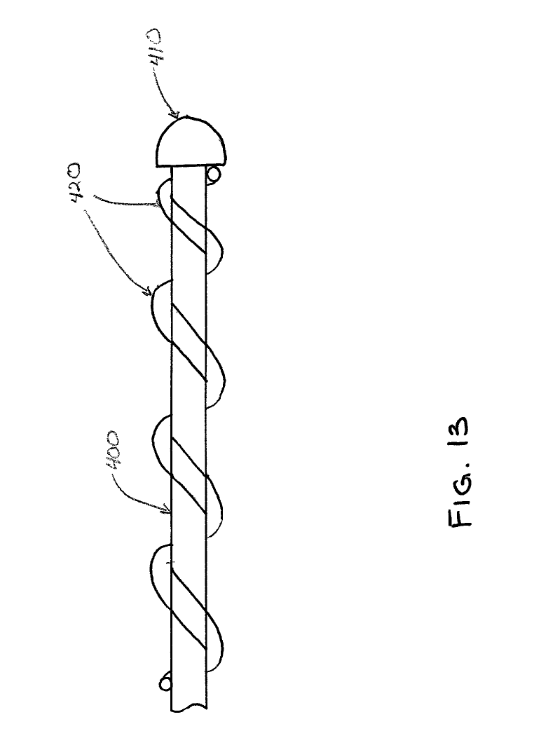

FIG. 13 shows an alternative embodiment of the invention. The applicator 284 is comprised of a solid applicator rod 400, preferably having a rod cap 410 at its distal end. An application wire structure 420 is coiled around the length of solid applicator rod 400. In one embodiment, application wire structure 420 is a metal wire that may be coiled, perforated or porous. In another embodiment, application wire structure 420 is comprised of a polymeric multi-filament cord.

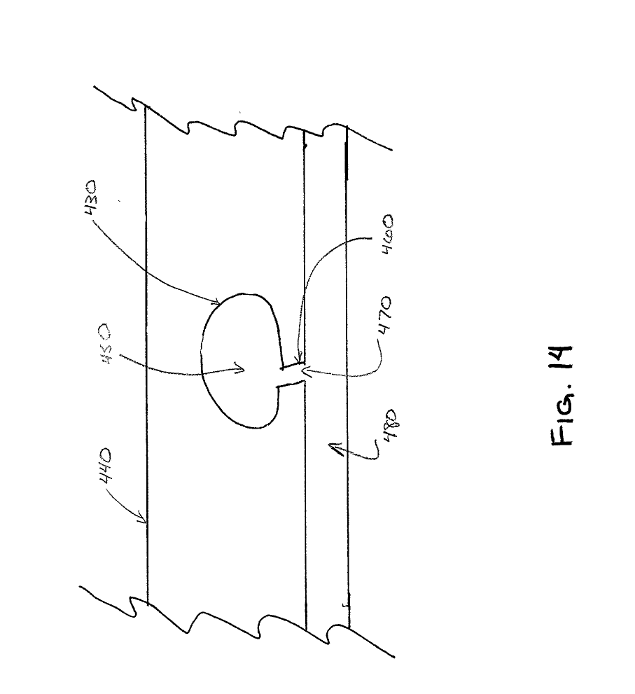

FIG. 14 shows a cross-section of a pseudoaneurysm. Pseudoaneurysm 430 is located below the surface of the skin 440. Blood pool 450 is supplied via pseudoaneurysm neck 460. Pseudoaneurysm neck 460 is located at injury site 470 of blood vessel 480.

In constructing the embodiments of the invention, materials appropriate to percutaneous devices should be used. For piston housing components including the proximal shaft 10, mid shaft 20, piston housing 100 and piston body cap 130 appropriate materials of construction include: fluoropolymers, Acetil (Delrin), other low friction polymers or other suitable polymers. Components to be gripped or biased by the user including proximal slide 30, distal slide 40, finger wings 50, luer tip 60 slidable piston handle 110, piston handle grip 120 and piston grip 150 will optimally be constructed of polycarbonate or other stiff, rigid polymer materials. The hollow needle 70 is optimally constructed of an appropriate grade of stainless steel. The thrombin delivery components including the piston 80, stylus 90, spring coil 160, and other delivery embodiments are optimally constructed of superelastic Nitinol, a combination of Nitinol and stainless steel or an appropriate grade of stainless steel.

In practice, the preferred embodiment is used following the location and diagnosis of a pseudoaneurysm 430. The medical personnel acquire wetting agent vial 180. Hollow needle 70 is inserted through rubber septum 190 and into wetting agent 200. Piston 80 is extended into wetting agent 200 by biasing proximal slide 30 and distal slide 40. Stylus 90 comes into contact with wetting agent 200. Wetting agent 200 coats the interstitial spaces 170 between in distal projection 90. Piston 80 and distal projection 90 are retracted within the hollow needle 70 by biasing proximal slide 30 and distal slide 40. Hollow needle 70 is withdrawn through the rubber septum 180.

The medical personnel then acquire dry clotting agent vial 210. Hollow needle 70 is inserted through rubber septum 220, either directly or coaxially through a cannula 222, and into dry clotting agent 230. Piston 80 is extended into dry clotting agent 230 by biasing proximal slide 30 and distal slide 40. Distal projection 90 comes into contact with dry clotting agent 230. Dry clotting agent 230 coats the now wetted interstitial spaces 170 in coiled wire 90. Piston 80 and distal projection 90 are retracted with the hollow needle 70 by biasing proximal slide 30 and distal slide 40. Preferably, cannula 222 is used to prevent the dulling of needle 70 as the needle is inserted through the rubber septum 220 or 190 and is also provided with an appropriate standoff height to prevent the tip of the needle 70 from bottoming out in the vial 210 or 180. Once distal projection 90 is retracted, the hollow needle 70 is withdrawn through rubber septum 220.

Gripping finger wing 50, the medical personnel insert hollow needle 70 through the patient's skin 440 until hollow needle 70 has accessed pseudoaneurysm 430. Proximal slide 30 and distal slide 40 are biased and piston 80 along with distal projection 90 emerge from hollow needle 70 and into blood pool 450 of pseudoaneurysm 430. Dry clotting agent 230 retained in interstitial spaces 170 causes thrombosis and clotting to occur inside blood pool 450 on the surface of distal projection 90. Following clot formation, proximal slide 30 and distal slide 40 are biased causing piston 80 and distal projection 90 to be withdrawn through hollow needle 70. As distal projection 90 enters hollow needle 70, the blood clot is removed from coils 160 and is left under skin 440 for resorption into patient's body. Using finger wing 50, the medical personnel remove hollow needle 70 from the patients skin 440.

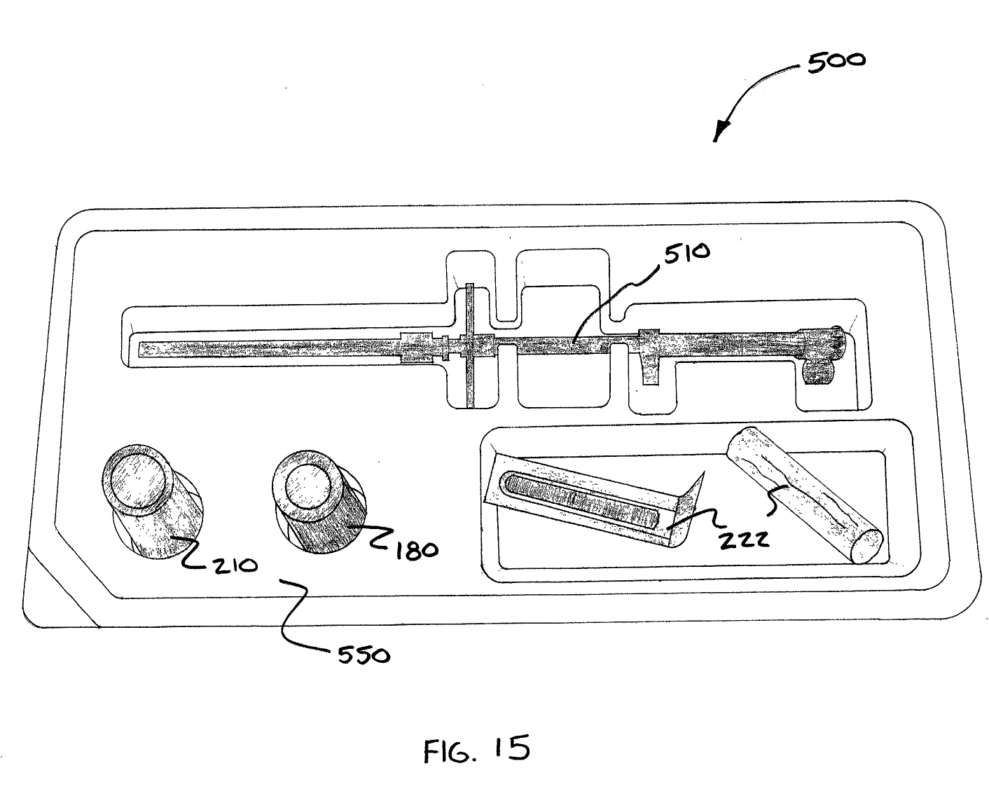

Referring to FIG. 15, a kit 500 can be provided that includes an apparatus 510 having a proximal handle 520 and a distal portion 530 having a needle 70 with a distal projection 90, a wetting agent vial 180, a dry clotting agent vial 210 and a pair of cannulas 222 contained within a tray 550 that is sterilized and sealed, preferably by a pealable sheeting material 560. The apparatus 510 may be any of the embodiments of the invention as previously described. The vials 180 and 210 are used in the manner as has been described, with the vial 210 containing sufficient clotting agent 230 necessary for treating a single patient. Preferably, the amount of clotting agent 230 in kit 500 is sufficient to treat any size pseudoaneurysm. Alternatively, different kits 500 may be provided having different sizes or arrangements of apparatus 510, or having vials 210 containing different quantities or compositions of clotting agents 230. Preferably, tray 550 and sheeting material 560 are plastic materials and tray 550 includes corresponding compartments individually associated with each of the items included in kit 500. In one embodiment, the needle 70 is contained in a separate compartment from the proximal portion 510 of the apparatus 500. In another embodiment, a separate stylet can be provided as part of the components of the kit 500. It will be recognized that numerous alternative sterile packaging arrangements known in the art could also be utilized to create kit 500.

Though a complete description of the preferred embodiment is provided, this is in no way meant to limit the application of the present invention. To one schooled in the art, it is clear that the embodiments contained in the remaining figures could be similarly used to accomplish thrombosis of a pseudoaneurysm. It is also clear that many types and forms of clotting agents could be used in conjunction with the present invention. These hemostatic agents include but are not limited to natural or synthetic compounds including modified derivatives such as thrombin, collagen, fibrinogen, oxidized cellulose, gelatin, chitosan, alginic acid, astringents such as tannic acid and vasoconstrictors such as epinephrine and angiotensin II or any combination of the above.

While the present invention is directed most particularly to pools of blood in the form of pseudoaneurysms, it will be understood that the present invention is capable of directly percutaneously treating any volume of blood within a patient for the purpose of forming a clot in that volume of blood. Examples of such other volumes of blood treatable by the present invention include aneurysms, hematomas, endoleaks or even a quantity of flowing blood.

What is claimed:

1. An apparatus for treating a pseudoaneurysm with an aliquot of a clotting agent, the apparatus comprising:
an applicator having a distal portion adapted to be percutaneously inserted into the pseudoaneurysm, the distal portion having at least one retaining structure defined on at least one surface of the distal portion that collectively retains the aliquot of the clotting agent as the distal portion is inserted into the pseudoaneurysm and exposes the aliquot of the clotting agent to blood within the pseudoaneurysm to initiate a clotting cascade, the at least one retaining structure arranged on the at least one surface such that the clotting cascade initiates at least one clot and that the aliquot of the clotting agent remains operably associated with the applicator until such time as the distal portion of the applicator is removed from the pseudoaneurysm in such a manner as to leave the at least one clot within the pseudoaneurysm.

2. The apparatus of claim 1 wherein the distal portion includes at least one spring member and the at least one retaining structure comprises adjacent coils of the at least one spring member with the aliquot of the clotting agent retained in interstitial spaces defined between the adjacent coils.

3. The apparatus of claim 2 wherein the apparatus includes an outer sleeve coaxial with the at least one spring member and a plunger member operably engaged with the at least one spring member to selectively extend the at least one spring member beyond a distal end of the outer sleeve.

4. The apparatus of claim 3 wherein the at least one spring member is preformed to assume a coiled configuration when the spring member is extended beyond the distal end of the outer sleeve.

5. The apparatus of claim 1 wherein the clotting agent is a wettable clotting agent and the at least one retaining structure includes a roughened, irregular surface to facilitate retention of the wettable clotting agent.

6. A system for treating a pseudoaneurysm comprising:
a stylet adapted to create an entry in skin overlying the pseudoaneurysm;
a blood clotting agent; and
an applicator having a distal portion adapted to be percutaneously inserted into the pseudoaneurysm through the entry created by the stylet, the distal portion having at least one retaining structure defined on at least one surface of the distal portion that collectively retains the clotting agent as the distal portion is inserted into the pseudoaneurysm and exposes the clotting agent to blood within the pseudoaneurysm to initiate a clotting cascade that creates at least one clot,
such that the clotting agent remains operably associated with the applicator until such time as the distal portion of the applicator is removed from the pseudoaneurysm in such a manner as to leave the at least one clot within the pseudoaneurysm.

7. A method for a user to treat a pseudoaneurysm with an aliquot of a clotting agent, the method comprising:
providing an applicator having a portion distal to the portion grasped by the user, the distal portion having at least one retaining structure defined on at least one surface of the distal portion that collectively retains the aliquot of the clotting agent as the distal portion is inserted into the pseudoaneurysm;
associating the aliquot of the clotting agent with the at least one retaining structure;
creating an entry for introducing at least a portion of the distal portion of the applicator into the pseudoaneurysm;
introducing at least a portion of the distal portion of the applicator into the pseudoaneurysm thereby exposing at least a portion of the aliquot of the clotting agent to blood in the pseudoaneurysm and thereby initiating a clotting cascade such that there is created at least one clot operably associated with at least a portion of the distal portion of the applicator;
waiting until the clotting cascade has progressed sufficiently to make the clot larger than the entry; and
withdrawing the distal portion of the applicator from the pseudoaneurysm so as to leave the clot within the pseudoaneurysm.

8. The method of claim 7 further comprising the step of:
providing an applicator wherein the distal portion includes at least one spring member and the at least one retaining structure comprises adjacent coils of the at least one spring member; and, associating the aliquot of the clotting agent in interstitial spaces defined between the adjacent coils.

9. The method of claim 8 further comprising the step of:
providing an outer sleeve that is coaxial with the at least one spring member and an extending means operably engaged with the at least one spring member to selectively extend the at least one spring member beyond a distal end of the outer sleeve;
positioning the distal portion of the outer sleeve so the spring member may be extended beyond the distal end of the outer sleeve to expose at least some of the clotting agent to blood in the pseudoaneurysm; and
extending the spring member beyond the distal end of the outer sleeve to expose at least some of the clotting agent to blood in the pseudoaneurysm.

10. A method for treating a pseudoaneurysm comprising:
providing a blood clotting agent and a device comprising a stylet and an applicator, with the stylet being adapted to create an entry in skin overlying the pseudoaneurysm and the applicator having a distal portion adapted to be percutaneously inserted into the pseudoaneurysm through the entry created by the stylet; the distal portion having at least one retaining structure defined on at least one surface of the distal portion that collectively retains the clotting agent;
associating the clotting agent with at least one receptacle;
using the stylet to create an entry for introducing at least a portion of the distal portion of the applicator into the pseudoaneurysm;
introducing at least a portion of the distal portion of the applicator into the pseudoaneurysm thereby exposing at least a portion of the aliquot of the clotting agent to blood in the pseudoaneurysm and thereby initiating a clotting cascade such that there is created at least one clot operably associated with at least a portion of the distal portion of the applicator;
waiting until the clotting cascade has progressed sufficiently to make the clot larger than the entry; and
withdrawing the distal portion of the applicator from the pseudoaneurysm so as to leave the clot within the pseudoaneurysm.

11. The method of claim 10 further comprising the step of:
providing an applicator wherein the distal portion includes at least one spring member and the at least one retaining structure comprises adjacent coils of the at least one spring member; and,
associating the aliquot of the clotting agent in interstitial spaces defined between the adjacent coils.

12. The method of claim 11 further comprising the step of:
providing an outer sleeve that is coaxial with the at least one spring member and an extending means operably engaged with the at least one spring member to selectively extend the at least one spring member beyond a distal end of the outer sleeve; and,
positioning the distal portion of the outer sleeve so the spring member may be extended beyond the distal end of the outer sleeve to expose at least some of the clotting agent to blood in the pseudoaneurysm;
extending the spring member beyond the distal end of the outer sleeve to expose at least some of the clotting agent to blood in the pseudoaneurysm.

13. A device for treating a subcutaneous pool of blood, the device comprising;
a blood clotting agent; and
an applicator adapted to be introduced into the subcutaneous pool of blood with a surface adapted to be exposed the blood, the surface having means for associating the blood clotting agent with the surface so that the blood clotting agent remains associated with the surface while the surface is exposed to the blood.

14. A kit for treating a pseudoaneurysm comprising:
a tray adapted to be sealed in a sterile manner;
a first vial containing a wetting agent positioned in the tray;
a second vial containing a blood clotting agent positioned in the tray; and
an applicator having a distal portion adapted to be percutaneously inserted into the pseudoaneurysm, the distal portion having at least one retaining structure defined on at least one surface of the distal portion that collectively retains a quantity of the clotting agent when the distal portion is first inserted into and withdrawn from the first vial and then inserted into and withdrawn from the second vial.

15. The kit of claim 14 wherein the distal portion of the applicator includes at least one spring member and the at least one retaining structure comprises adjacent coils of the at least one spring member with the quantity of the clotting agent retained in interstitial spaces defined between the adjacent coils.

16. The kit of claim 15 wherein the applicator includes an outer sleeve coaxial with the at least one spring member and a plunger member operably engaged with the at least one spring member to selectively extend the at least one spring member beyond a distal end of the outer sleeve.

17. The kit of claim 16 wherein the at least one spring member is preformed to assume a coiled configuration when the spring member is extended beyond the distal end of the outer sleeve.

18. The kit of claim 14 wherein the at least one retaining structure includes a roughened, irregular surface to facilitate retention of the wettable clotting agent.

19. The kit of claim 14 wherein the first and second vials include a seal and wherein the kit further comprises at least one cannula positioned in the tray, the cannula having a tip that pierces the seal and a standoff structure that prevents the distal portion of the applicator from advancing into the vial more than a predetermined distance.
